(19) United States
(12) Patent Application Publication
(10) Pub. No.: US 2024/0132422 A1
(43) Pub. Date: Apr. 25, 2024

(54) Spin-Labeled Nucleosides and Phosphoramidites

(71) Applicant: Somalogic Operating Co., Inc., Boulder, CO (US)

(72) Inventors: Catherine R. Fowler, Longmont, CO (US); John C. Rohloff, Las Vegas, NV (US); Brian E. Ream, Louisville, CO (US)

(73) Assignee: Somalogic Operating Co., Inc., Boulder, CO (US)

(21) Appl. No.: 18/477,649

(22) Filed: Sep. 29, 2023

Related U.S. Application Data
(60) Provisional application No. 63/412,802, filed on Oct. 3, 2022.

(51) Int. Cl. C07B 59/00; C07H 19/067; C07H 19/073; C07H 23/00

(52) U.S. Cl. CPC C07B 59/005; C07H 19/067; C07H 19/073; C07H 23/00; C07B 2200/05

(57) ABSTRACT

Spin-labeled uridine nucleosides and phosphoramidites are provided, including improved methods of synthesis and oligonucleotides comprising the spin-labeled nucleosides.

### CROSS-REFERENCE TO RELATED APPLICATIONS

This application claims the benefit of priority of U.S. Provisional Application No. 63/412,802, filed on Oct. 3, 2022, which is incorporated herein by reference in its entirety for any purpose.

### FIELD

This disclosure relates to the field of spin-labeled nucleosides and phosphoramidites, including improved methods of synthesis and oligonucleotides comprising the spin-labeled nucleosides.

### BACKGROUND

Quantum sensing based on nitrogen vacancy (NV) centers in diamond has emerged as a powerful technology that enables the detection of individual proteins and DNA molecules. A NV center can detect binding through a shift in the transition frequency of a spin-labeled oligonucleotide. Further, the detection of a binding event at a single-molecule level via an electron paramagnetic resonance measurement (EPR) signature would remove the ambiguity associated with non-specific adsorption in existing fluorescent techniques.

There remains a need in the art for alternative spin-labeled bases, nucleosides, oligonucleotides, and phosphoramidites to enable quantum sensing of, for example, binding events. The techniques described herein provide methods for the introduction of a spin-labeled group to any site within an oligonucleotide via a modified deoxyuridine phosphoramidite. As the chemistry involved in production of synthetic DNA and RNA will affect the free radical in the spin label, an important factor in using this approach is the transient protection of the radical group. This disclosure provides two different protection strategies for preserving the masked radical during monomer production and subsequent synthesis of a modified oligonucleotide, allowing for the unmasking of the radical group using means compatible with the properties of oligonucleotides.

### SUMMARY

In some embodiments, a compound having the structure

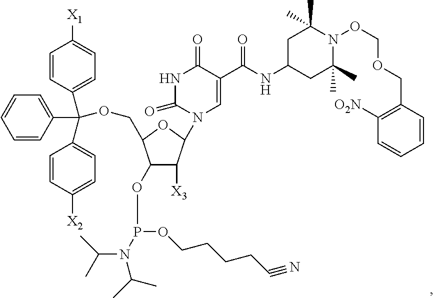

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy.

In some embodiments, X₁ and X₂ are both methoxy. In some embodiments, X₃ is hydrogen. In some embodiments, X₃ is methoxy. In some embodiments, X₃ is fluoro. In some embodiments, X₃ is tert-butyldimethylsilyloxy.

In some embodiments, a compound provided herein is selected from:

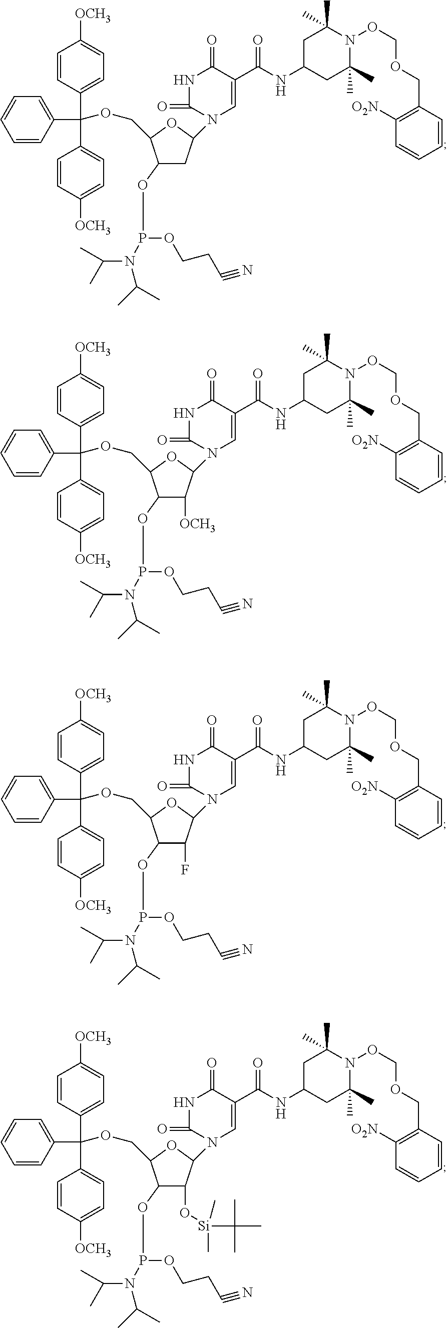

and salts thereof.

In some embodiments, a compound having the structure

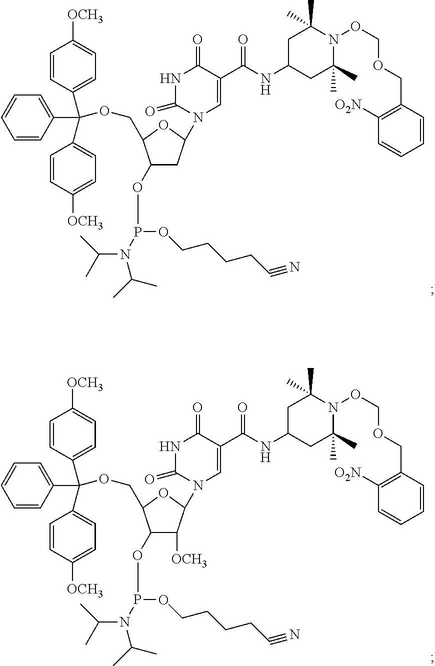

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy.

In some embodiments, X₁ and X₂ are methoxy. In some embodiments, X₃ is hydrogen. In some embodiments, X₃ is methoxy. In some embodiments, X₃ is fluoro. In some embodiments, X₃ is tert-butyldimethylsilyloxy.

In some embodiments, the compound is selected from:

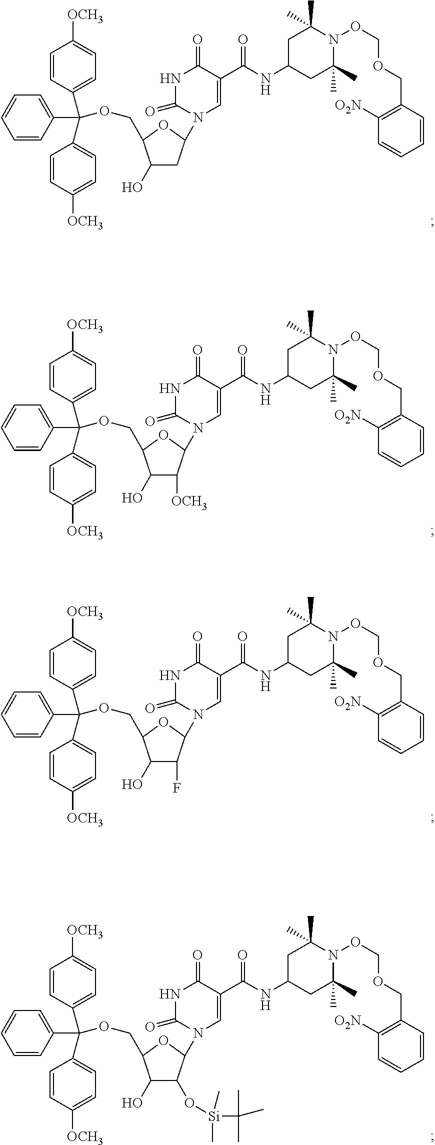

and salts thereof.

In some embodiments, a method of producing a compound having the structure:

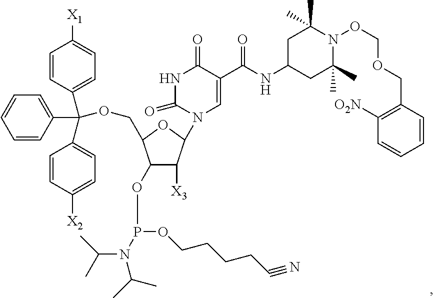

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy. In some embodiments, the method comprising reacting the compound

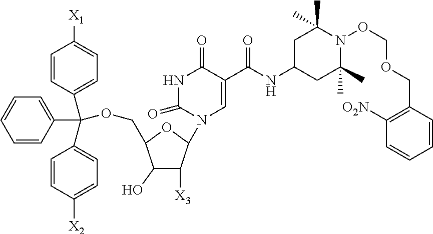

with 2-cyanoethyl N,N,N′,N′-tetraisopropylphosphorodiamidite and pyridine trifluoroacetic acid in dichloromethane, 2-cyanoethyl N,N-diisopropyl chlorophosphoramidite and diisopropylethylamine in dichloromethane, or similar conditions.

In some embodiments, the method produces a compound selected from:

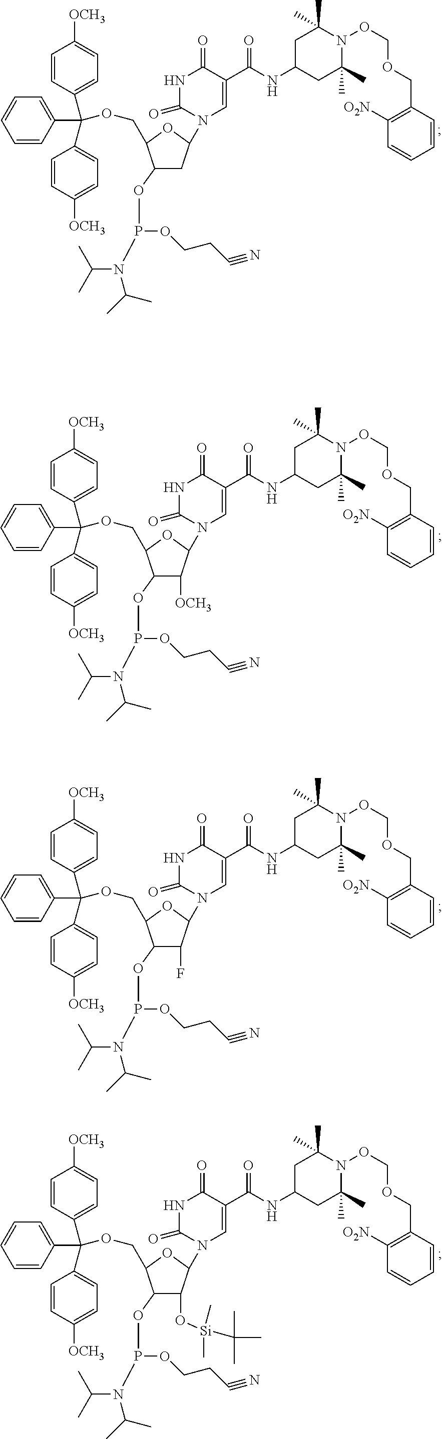

and salts thereof.

In some embodiments, a method of producing a compound having the structure:

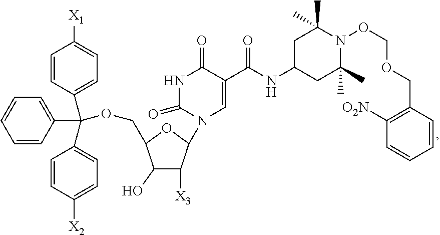

or a salt thereof, is provided. In some embodiments, X₁ and X₂ are each independently selected from methoxy and hydrogen. In some embodiments, X₃ is selected from a methoxy, fluoro, hydrogen, and tert-butyldimethylsilyloxy. In some embodiments, the method comprises reacting the compound

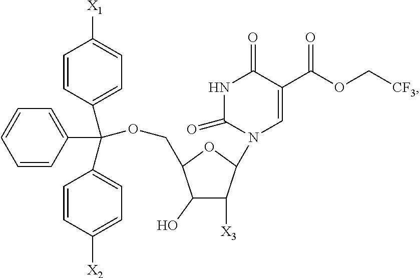

or a salt thereof, with the compound

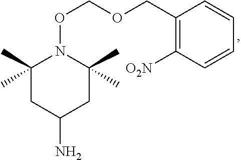

prepared according to previously reported literature (Weinrich et al. Chem.-Eur. J. 2018, 24, 6202-6207).

In some embodiments, the method produces a compound selected from:

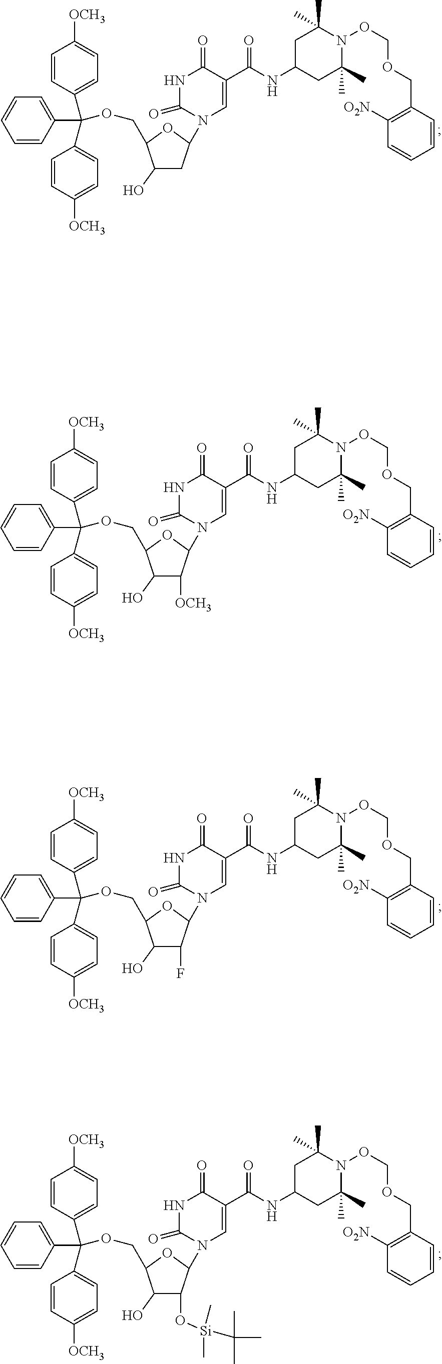

and salts thereof.

In some embodiments, a method of producing a compound having the structure:

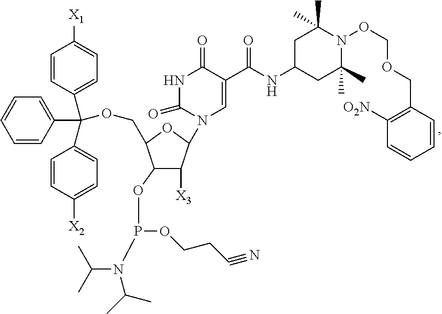

or a salt thereof, is provided,
wherein,
X₁ and X₂ are each independently selected from methoxy and hydrogen; and
X₃ is selected from a methoxy, fluoro, hydrogen, and tert-butyldimethylsilyloxy; comprising the steps of:
a) reacting the compound

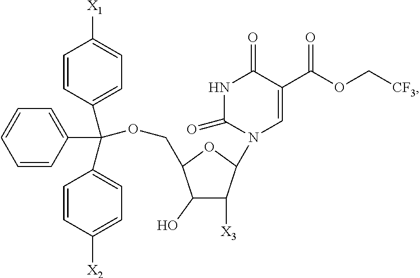

prepared according to previously reported literature (Nomura et al. Nucleic Acids Research, 1997, 25, 2784-2791; Ito et al. Nucleic Acids Research, 2003. 25. 2514-25231. with the compound

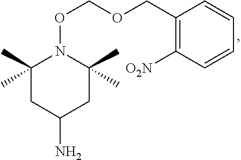

prepared according to previously reported literature (Weinrich et al. Chem.-Eur. J. 2018. 24. 6202-62071. to form the compound

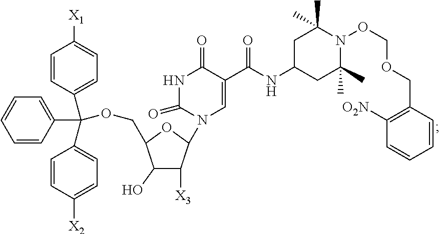

and
b) reacting the compound

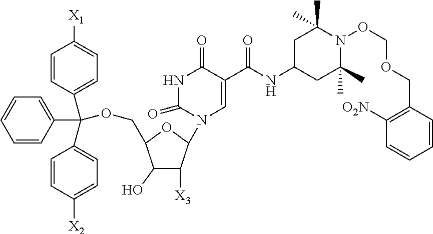

with 2-cyanoethyl N,N,N′,N′-tetraisopropylphosphorodiamidite and pyridine trifluoroacetic acid in dichloromethane, 2-cyanoethyl N,N-diisopropyl chlorophosphoramidite and diisopropylethylamine in dichloromethane, or similar conditions.

In some embodiments, the method produces a compound selected from:

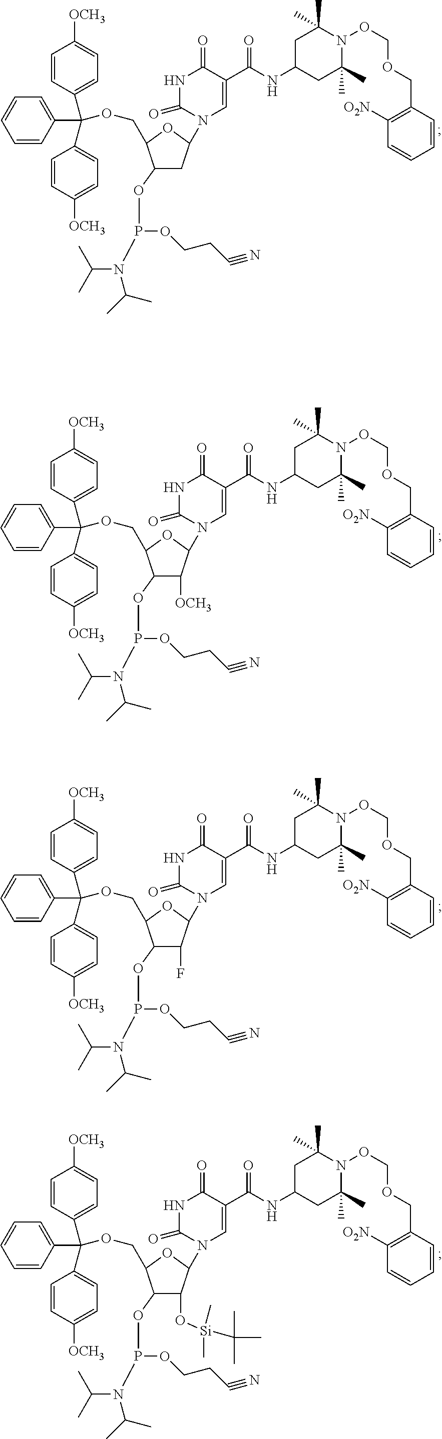

and salts thereof.

In embodiments, oligonucleotides are provided, comprising at least one spin-labeled nucleotide, wherein at least one spin-labeled nucleotide in the oligonucleotide has the structure:

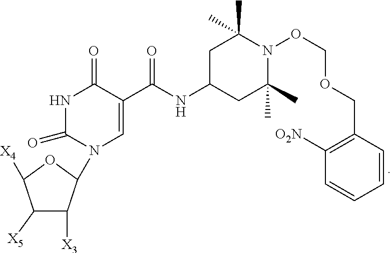

In some embodiments, X₃ is selected from a methoxy, fluoro, hydrogen, and tert-butyldimethylsilyloxy. In some embodiments, X₄ is selected from OH, —OR′, —SR′, and -Z-P(Z′)(Z″)O—R″, wherein Z, Z′, and Z″ are each independently selected from O and S, R′ is H or a cap, and R″ is H, a cap, or an adjacent nucleotide. In some embodiments, X₅ is selected from —O-ss, —OR′, —SR′, and -Z-P(Z′)(Z″)O—R″, wherein ss is a solid support, Z, Z′, and Z″ are each independently selected from O and S, R′ is H or a cap, and R″ is H, a cap, or an adjacent nucleotide. In some embodiments, the solid support is controlled-pore glass (CPG). In some embodiments, Z′ is S and Z″ is O. In some embodiments, Z′ and Z″ are O.

In embodiments, oligonucleotides are provided, comprising at least one spin-labeled nucleotide, wherein at least one spin-labeled nucleotide in the oligonucleotide has the structure:

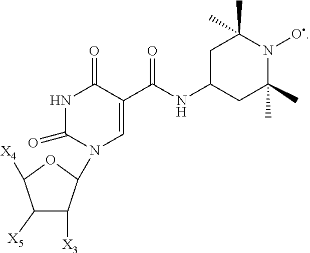

In some embodiments, X₃ is selected from a methoxy, fluoro, hydrogen, and tert-butyldimethylsilyloxy. In some embodiments, X₄ is selected from OH, —OR′, —SR′, and -Z-P(Z′)(Z″)O—R″, wherein Z, Z′, and Z″ are each independently selected from O and S, R′ is H or a cap, and R″ is H, a cap, or an adjacent nucleotide. In some embodiments, X₅ is selected from —O-ss, —OR′, —SR′, and -Z-P(Z′)(Z″)O—R″, wherein ss is a solid support, Z, Z′, and Z″ are each independently selected from O and S, R′ is H or a cap, and R″ is H, a cap, or an adjacent nucleotide. In some embodiments, the solid support is controlled-pore glass (CPG). In some embodiments, Z′ is S and Z″ is O. In some embodiments, Z′ and Z″ are O.

In some embodiments, a method of producing an oligonucleotide comprising at least one spin-labeled nucleotide is provided, comprising incorporating at least one nucleotide having the structure:

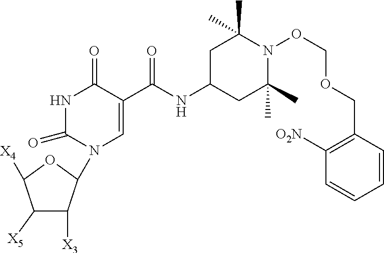

into a nucleotide sequence on a solid support; and removing the

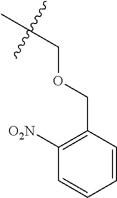 [SMILES: *COCc1ccccc1[N+](=O)[O-]]

protecting group from the at least one spin-labeled nucleotide incorporated into the oligonucleotide. In some embodiments, X₃ is selected from a methoxy, fluoro, hydrogen, and tert-butyldimethylsilyloxy. In some embodiments, X₄ is selected from OH, —OR′, —SR′, and -Z-P(Z′)(Z″)O—R″, wherein Z, Z′, and Z″ are each independently selected from O and S, R′ is H or a cap, and R″ is H, a cap, or an adjacent nucleotide. In some embodiments, X₅ is selected from —O-ss, —OR′, —SR′, and -Z-P(Z′)(Z″)O—R″, wherein ss is a solid support, Z, Z′, and Z″ are each independently selected from O and S, R′ is H or a cap, and R″ is H, a cap, or an adjacent nucleotide. In some embodiments, the solid support is controlled-pore glass (CPG). In some embodiments, Z′ is S and Z″ is O. In some embodiments, Z′ and Z″ are O.

### BRIEF DESCRIPTION OF THE DRAWINGS

FIG. 1 shows chromatograms of a deprotected oligonucleotide containing protected TEMPO and the same oligonucleotide after photo-cleavage of the protecting group.

### DETAILED DESCRIPTION

The techniques described herein provide methods for the introduction of a spin-labeled group to any site within an oligonucleotide via a modified deoxyuridine phosphoramidite. As the chemistry involved in production of synthetic DNA and RNA may affect the free radical in the spin label, one aspect of this approach is the transient protection of the radical group. This disclosure provides two different protection strategies for preserving the masked radical during monomer production and subsequent synthesis of a modified oligonucleotide, allowing for the unmasking of the radical group using means compatible with the properties of oligonucleotides.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “ an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean ±20% of the indicated range, value, or structure, unless otherwise indicated. As used herein, the terms “include” and “comprise” are open ended and are used synonymously.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs).

As used herein, the term “TEMPO dU” is used to generally refer to uridylyl nucleotides comprising a 5-position 4-Amino-2,2,6,6-tetramethylpiperidine-1-oxyl free radical. Use of the term “TEMPO dU” is not intended to be limiting with regard to the 2′ position of the ribose, and the term should be construed to include, but not be limited to, nucleotides comprising, for example, —H, —OH, —OMe, or —F at the 2′-position, unless a particular 2′ moiety is indicated.

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers, but the terms nucleic acid, oligonucleotide, and polynucleotide are not limited to aptamers.

As used herein, the term “at least one nucleotide” when referring to modifications of a nucleic acid, refers to one, several, or all nucleotides in the nucleic acid, indicating that any or all occurrences of any or all of A, C, T, G or U in a nucleic acid may be modified or not.

As used herein, a “phorphoramidite” is a nucleotide comprising a

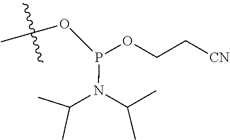 [SMILES: *OP(OCCC#N)N(C(C)C)C(C)C]

group attached to the 3′ carbon of the ribose, or an equivalent position on another sugar moiety. In some embodiments, a phosphoramidite comprises a protecting group on the 5′-OH of the ribose, such as a trityl protecting group, for example, a dimethoxytrityl protecting group.

As used herein, “solid phase synthesis” refers to solid-phase oligonucleotide synthesis using phosphoramidite chemistry, unless specifically indicated otherwise.

### Compounds

The present disclosure provides the compounds shown in Table A, as well as salts thereof, and methods of making and using the compounds.

TABLE A
Compounds of the disclosure

| Compound | Name | Structure |
|---|---|---|
| 1 | oNB-TEMPOdU CEP | 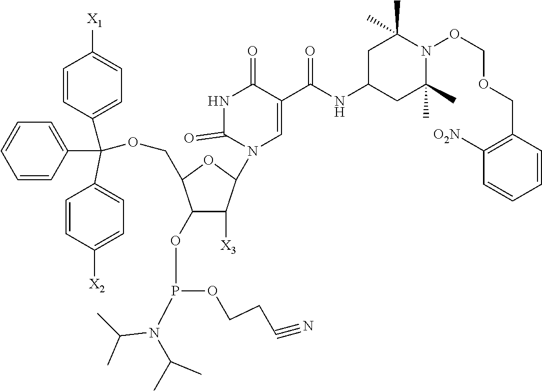 |
| 2 | DMT-oNB- TEMPOdU | 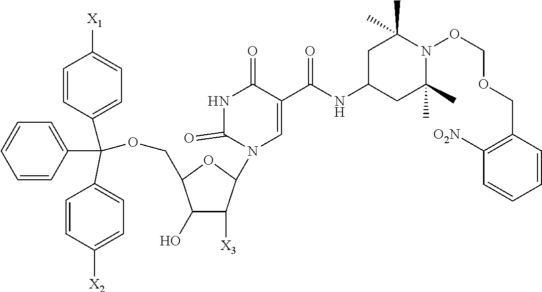 |

X₃ in the structures in Table A may, in some embodiments, be selected from methoxy, fluoro, hydrogen, and tert-butyldimethylsilyloxy. In some embodiments, compounds 1 to 4 in Table A may be used in solid-phase oligonucleotide synthesis to produce oligonucleotides comprising one or more spin-labeled nucleotides. Also provided herein are compounds comprising a structure selected from compounds 5 to 8, wherein the 3′ carbon of the ribose is linked to a solid phase, such as controlled-pore glass, through a linker moiety. In some embodiments, the 3′ carbon of the ribose is linked to a solid phase through a linker moiety selected from succinate, diglycolate, and alkylamino.

The compounds in Table A may be synthesized, in some embodiments, using the methods described herein, such as in the Examples herein.

### Salts

It may be convenient or desirable to prepare, purify, and/or handle a corresponding salt of the compound.

For example, if the compound is anionic, or has a functional group which may be anionic (e.g., —COOH may be —COCO⁻, then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R^{X+}, NH₂R^{X}₂⁺, NHR^{X}₂⁺, NR^{X}₄⁺). Examples of some suitable substituted ammonium ions are those derived from: ethylamine, diethylamine, dicyclohexylamine, triethylamine, butylamine, ethylenediamine, ethanolamine, diethanolamine, piperizine, benzylamine, phenylbenzylamine, choline, meglumine, and tromethamine, as well as amino acids, such as lysine and arginine. An example of a common quaternary ammonium ion is N(CH₃)₄⁺.

If the compound is cationic or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Modified Oligonucleotides

As used herein, the terms “modify,” “modified,” “modification,” and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. Additional modifications can include backbone modifications, methylations, unusual base-pairing combinations such as the isobases isocytidine and isoguanidine, and the like. Modifications can also include 3′ and 5′ modifications, such as capping. Nonlimiting exemplary caps include 5′-trimethoxystilbene cap, 5′ pyrene cap, 5′ adenylated cap, 5′ guanosine triphosphate cap, 5′ N7-methyl guanosine triphosphate cap, and 3′ Uaq cap. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers.

Oligonucleotides can also contain analogous forms of ribose or deoxyribose sugars that are generally known in the art, including 2′-O-methyl, 2′-O-allyl, 2′-O-ethyl, 2′-O-propyl, 2′-O-CH₂CH₂OCH₃, 2′-fluoro, 2′-NH₂ or 2′-azido, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted herein, one or more phosphodiester linkages may be replaced by alternative linking groups. These alternative linking groups include embodiments wherein phosphate is replaced by P(O)S (“thioate”), P(S)S (“dithioate”), (O)NR^{X}₂ (“amidate”), P(O)R^{X}, P(O)OR^{X}′, CO or CH₂ (“formacetal”), in which each R^{X} or R^{X}40 are independently H or substituted or unsubstituted alkyl (C1-C20) optionally containing an ether (—O—) linkage, aryl, alkenyl, cycloalky, cycloalkenyl or araldyl. Not all linkages in an oligonucleotide need be identical. Substitution of analogous forms of sugars, purines, and pyrimidines can be advantageous in designing a final product, as can alternative backbone structures like a polyamide backbone, for example.

Oligonucleotides can also contain analogous forms of carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

If present, a modification to the nucleotide structure can be imparted before or after assembly of a polymer. A sequence of nucleotides can be interrupted by non-nucleotide components. An oligonucleotide can be further modified after polymerization, such as by conjugation with a labeling component.

### Preparation of Oligonucleotides

The automated synthesis of oligodeoxynucleosides is routine practice in many laboratories (see, e.g., Matteucci, M. D. and Caruthers, M. H., (1990) J. Am. Chem. Soc., 103:3185-3191, the contents of which are hereby incorporated by reference in their entirety). Synthesis of oligoribonucleosides is also well known (see e.g., Scaringe, S. A., et al., (1990) Nucleic Acids Res. 18:5433-5441, the contents of which are hereby incorporated by reference in their entirety). As noted herein, the phosphoramidites are useful for incorporation of the modified nucleoside into an oligonucleotide by chemical synthesis, and the triphosphates are useful for incorporation of the modified nucleoside into an oligonucleotide by enzymatic synthesis. (See e.g., Vaught, J. D. et al. (2004) J. Am. Chem. Soc., 126:11231-11237; Vaught, J. V., et al. (2010) J. Am. Chem. Soc. 132, 4141-4151; Gait, M. J. “Oligonucleotide Synthesis a practical approach” (1984) IRL Press (Oxford, UK); Herdewijn, P. “Oligonucleotide Synthesis” (2005) (Humana Press, Totowa, N.J. (each of which is incorporated herein by reference in its entirety).

In some embodiments, the compounds provided herein, and in particular, compounds of Table A, may be used in standard phosphoramidite oligonucleotide synthesis methods, including automated methods using commercially available synthesizers. Following synthesis, the protecting group on the TEMPO moiety is removed by exposing the oligonucleotide to UV light.

### EXAMPLES

The following examples are presented in order to more fully illustrate some embodiments of the invention. They should, in no way be construed, however, as limiting the broad scope of the invention. Those of ordinary skill in the art can readily adopt the underlying principles of this discovery to design various compounds without departing from the spirit of the current invention.

### Example S1. Photocleavable TEMPO Protection Using 5′-O-(4,4′-Dimethoxytrityl)-5-[N-(O-methoxynitrobenzyl-TEMPO) carboxamide]-2′-deoxyuridine (TEMPOdU) Derivatives

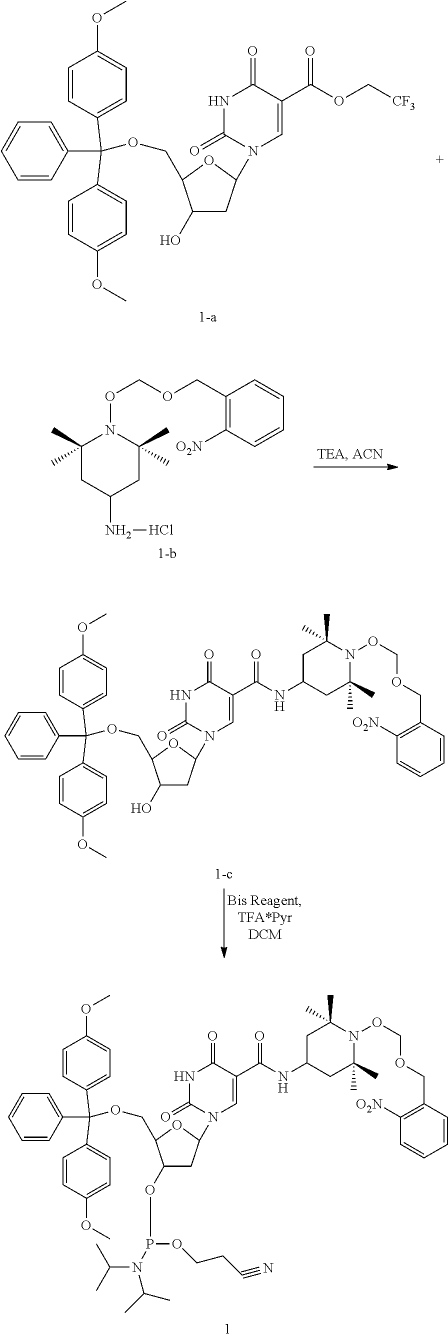

TEMPO was protected using a previously published strategy for placing a photolabile O-methoxynitrobenzyl on the oxygen radical of TEMPO amine. The protecting group was synthesized in several steps and attached to the oxygen radical of and N-protected TEMPO amine, after which the amine was deprotected to allow for further chemistries. The whole protected amine moiety was then reacted via carboxyamidation with 5′-O-dimethoxytrityl-5-trifluoroethoxycarbonyl-2′-deoxyuridine (TFEdU), which was prepared by the procedure of Matsuda et al (Noruma, Y.; Ueno, Y.; Matsuda, A. Nucleic Acids Research 1997, 25:2784-2791; Ito, T.; Ueno, Y.; Matsuda, A. Nucleic Acid Research 2003, 31:2514-2523). The resulting TEMPO-dU material was next converted to the corresponding phosphoramidite (amidite).

### Example S1.1 5′-O-(4,4′-Dimethoxytrityl)-5-[N-(O-methoxynitrobenzyl-TEMPO) carboxamide]-2′-deoxyuridine (1-c)

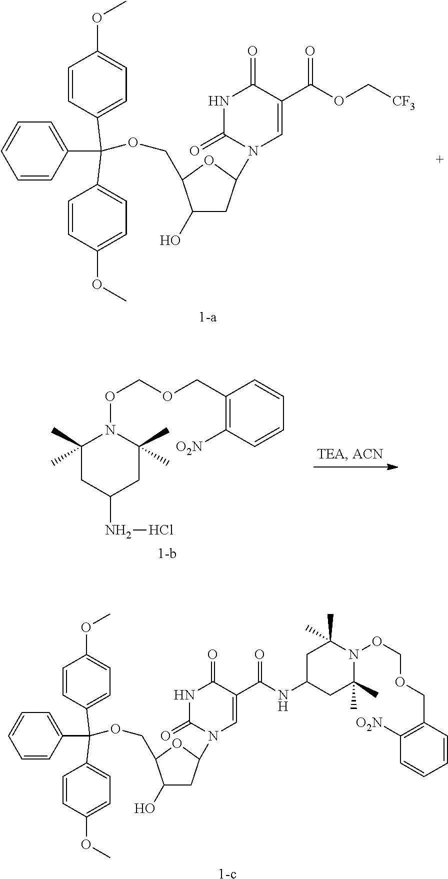

The starting material, 5′-O-dimethoxytrityl-5-trifluoroethoxycarbonyl-2′-deoxyuridine (1-a, 1.55 g, 2.36 mmol)) was charged into a dry, argon-purged round bottomed flask, protected from light. Dry acetonitrile (2.95 mL) and O-methoxynitrobenzyl TEMPO amine (previously synthesized) (1-b, 1.23 g, 2.83 mmol, 1.2 eq) were added to the flask and the mixture was stirred to dissolve the solids. Triethylamine (0.7 mL, 4.72 mmol, 2 eq) was added to the stirring mixture, after which the mixture was transferred to an oil bath and was heated under an inert atmosphere at 65° C. while stirring. Reaction progress was monitored by reversed phase HPLC (Waters 2795 HPLC with a 2489 detector and using a Waters Symmetry column, buffer A: 100 mM triethylammonium acetate, buffer B: acetonitrile, gradient: 70% buffer B, isocratic, over 30 minutes). After stirring 84 hours, analysis showed the reaction had reached an endpoint. The solvent was evaporated to recover a yellowish foam. The crude mixture was applied to a silica gel flash column equilibrated with 1% triethylamine/99% dichloromethane. The product was initially eluted with the 100% dichloromethane and then 99% dichloromethane/1% methanol to complete the elution. Product-containing fractions were concentrated to provide a white to off-white foam (1.02 g, 48.3% yield).

### Example S1.2 5′-O-(4,4′-Dimethoxytrityl)-4-N-acetyl-5-[N-(O-methoxynitrobenzyl-TEMPO) carboxamide]-2′-deoxyuridine-3′-O-(N,N-diisopropyl-O-2-cyanoethylphosphoramidite) (1)

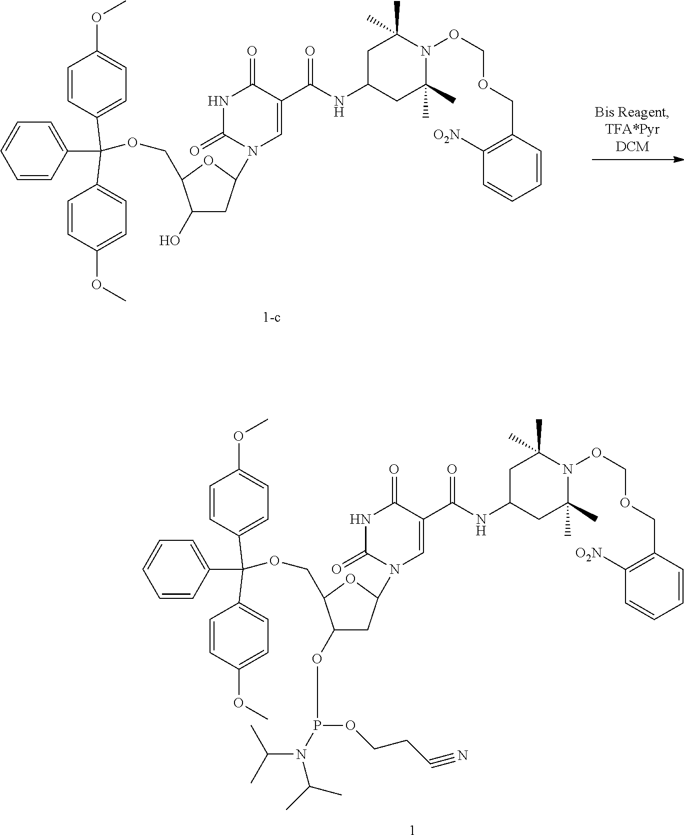

In a round-bottomed flask with magnetic stirring and protected from light, the product of the previous step (1-c, 1.00 g, 1.12 mmol) was dissolved in anhydrous dichloromethane (3.2 mL) under argon. To the reaction mixture was added 2-cyanoethyl-N,N,N′,N′-tetraisopropylphosphine (0.4 mL, 1.18 mmol, 1.05 eq) followed by pyridine trifluoroacetate (238 mg, 1.23 mmol, 1.1 eq). The reaction was stirred for 1 hour. The crude mixture was applied to a silica gel flash column conditioned with 50% ethyl acetate/49% hexanes/1% triethylamine and product elution was achieved using 50% ethyl acetate/50% hexanes and then 100% ethyl acetate. All mobile phases were chilled to 0° C. and sparged with argon and product was collected into argon-purged bottles. Product-containing fractions were concentrated to provide a white to off-white foam (0.74 g, 60% yield).

¹H-NMR (300 MHz, CD₃CN): δ=8.56 (d, J=6.9 Hz, 1H), 8.43/8.41 (s, 1H), 8.08 (dd, J_{A}=8.1 J_{B}=1.2 Hz, 1H), 7.79-7.84 (m, 1H) 7.74 (td, J_{A}=7.5 J_{B}=1.5 Hz, 1H), 7.50-7.85 (m, 1H), 7.41-7.48 (m, 2H), 7.18-7.39 (m, 7H), 6.85-6.93 (m, 4H), 6.13 (q, J=6.3Hz, 1H), 5.04 (d, J=9.3 Hz, 4H), 4.39-4.51 (m, 1H), 4.08-4.26 (m, 1H), 3.72-3.86 (m, 7H), 3.48-3.70 (m, 4H), 3.28-3.44 (m, 2H), 2.65 (t, J=6.0 Hz, 1H), 2.46-2.61 (m, 2H), 2.27-2.42 (m, 1H), 1.74-1.91 (m, 2H), 1.32-1.54 (m, 2H), 1.09-1.31 (m, 24H), 1.05 (d, J=6.9 Hz,3H).

³¹P-NMR (300 MHz, CD₃CN): δ=148.14/148.16 (s, 1P).

MS (m/z) calcd for C₅₇H₇₂N₇O₁₃P, 1094.2; found 1092.4 [M−H]⁻ (ESP⁻).

### Example S2. Synthesis of Oligonucleotides Comprising at Least One Protected Spin-Labeled Nucleotide

### Example S2.1 Solid-Phase Oligonucleotide Synthesis

An ABI 3900 automated DNA synthesizer (Applied Biosystems, Foster City, CA) was used with conventional phosphoramidite methods with minor changes to the coupling conditions for modified phosphoramidites. Modified phosphoramidites were used in 0.1 M solutions using acetonitrile with 0-40% dichloromethane and 0-20% sulfolane as the solvent. Solid support was an ABI style fritted column packed with controlled pore glass (CPG, LGC Biosearch Technologies, Petaluma CA) loaded with 3′-DMT-dT succinate with 1000 Å pore size. All syntheses were performed at the 50 nmol scale and the 5′ end of each sequence was modified with a hexaethyleneglycol spacer and biotin group for support attachment. Introduction of a TEMPO dU variant was carried out as a single-base replacement at select sites within the DNA strand using phosphoramidites synthesized using the methods described herein. Oligonucleotides were deprotected by treatment with concentrated ammonium hydroxide at 55° C. for 4-6 hours, after which the product mixtures were filtered and residual solvents removed in a Genevac HT-12 evaporator. Identity and percent full length product were determined using an Agilent 1290 Infinity with an Agilent 6130B single quadrupole mass spectrometry detector using an Acquity C18 column 1.7 μm 2.1×100 mm (Waters Corp, Milford, MA).

### Example S2.2 Post-Synthetic Processing of Protected TEMPO dU Oligonucleotides

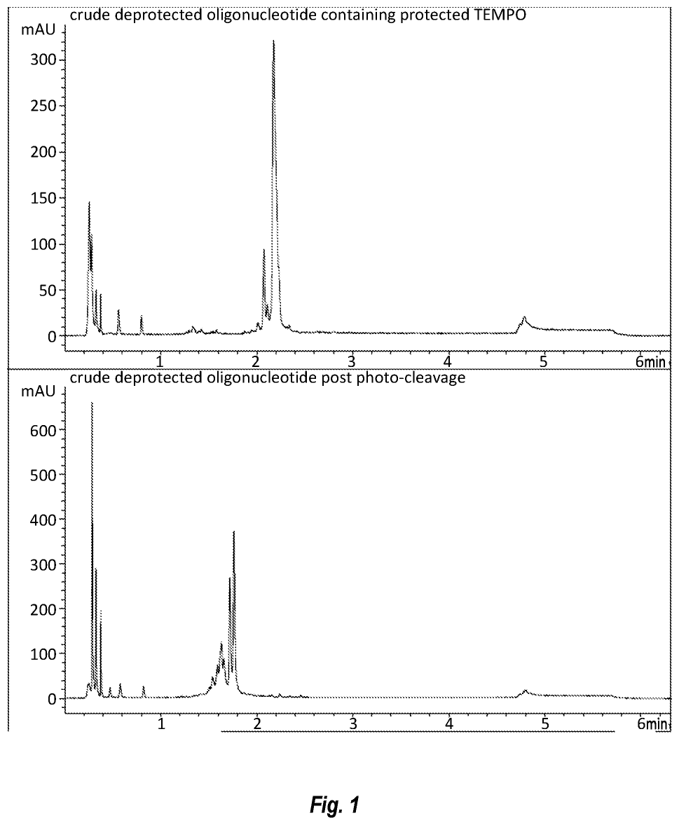

Following the post-deprotection evaporation of the oligonucleotide, the resulting crude residues were then redissolved in Water for Injection (WFI, HyPure WFI Quality Water, HyClone Laboratories, Logan, UT, or similar). Photo-cleavage of the O-methoxy-nitrobenzyl protecting group from the TEMPO moiety was effected via exposure to UV light (365 nm) for one hour. The crude material was then applied to a centrifugal filter (Millipore Amicon Ultra-15 3 kDa) and washed three times with 5 mL WFI per wash for removal of small molecule impurities. Product was collected in approximately 500 μL WFI without further purification. Identity and percent full length product were determined using an Agilent 1290 Infinity with an Agilent 6130B single quadrupole mass spectrometry detector using an Acquity C18 column 1.7 μm 2.1×100 mm (Waters Corp, Milford, MA). Chromatograms before and after photo-cleavage of the protecting group is shown in FIG. 1.

1. A method of producing the compound: 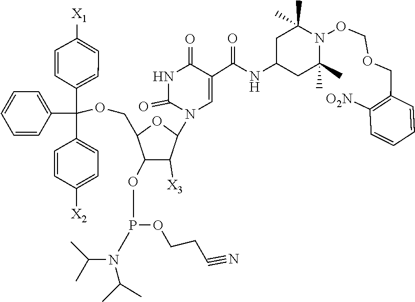
or a salt thereof,
wherein:
X₁ and X₂ are each independently selected from methoxy and hydrogen; and
X₃ is selected from methoxy, fluoro, hydrogen, and tert-butyldimethylsilyloxy;
the method comprising reacting the compound 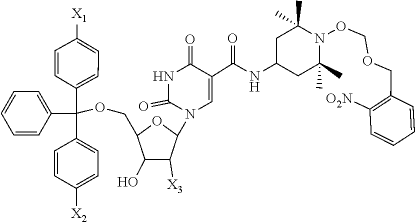
or a salt thereof, with 2-cyanoethyl N,N,N′,N′-tetraisopropylphosphorodiamidite and pyridine trifluoroacetic acid in dichloromethane, or 2-cyanoethyl N,N-diisopropyl chlorophosphoramidite and diisopropylethylamine in dichloromethane.

2. The method of claim 1, wherein the compound: 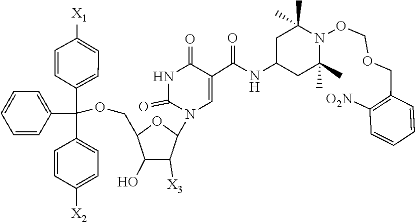
or a salt thereof, is prepared by reacting 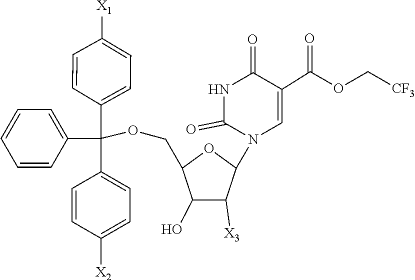
or a salt thereof, and 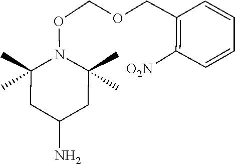
or a salt thereof, in the presence of triethylamine in acetonitrile.

3. A method of producing the compound: 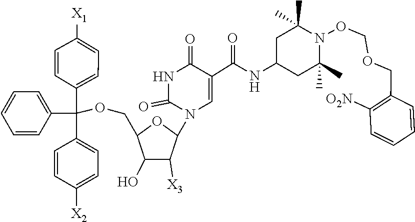
or a salt thereof,
wherein:
X₁ and X₂ are each independently selected from methoxy and hydrogen; and
X₃ is selected from methoxy, fluoro, hydrogen, and tert-butyldimethylsilyloxy;
the method comprising reacting 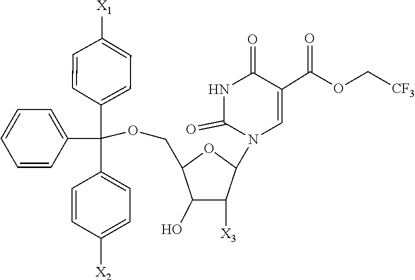
or a salt thereof, and 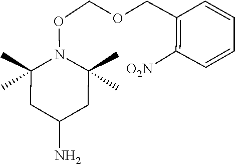
or a salt thereof, in the presence of triethylamine in acetonitrile.

4. The method of claim 1, wherein X₁ and X₂ are both methoxy.

5. The method of claim 1, wherein X₃ is hydrogen.

6. A compound selected from: 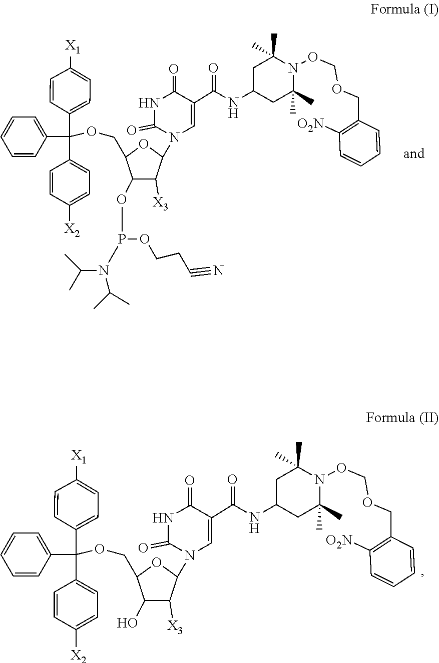
or a salt thereof,
wherein:
X₁ and X₂ are each independently selected from methoxy and hydrogen; and
X₃ is selected from methoxy, fluoro, hydrogen, and tert-butyldimethylsilyloxy.

7. (canceled)

8. The compound of claim 6, wherein X₁ and X₂ are both methoxy.

9. The compound of claim 6, wherein X₃ is hydrogen.

10. An oligonucleotide comprising at least one protected spin-labeled nucleotide, wherein at least one protected spin-labeled nucleotide in the oligonucleotide has the structure: 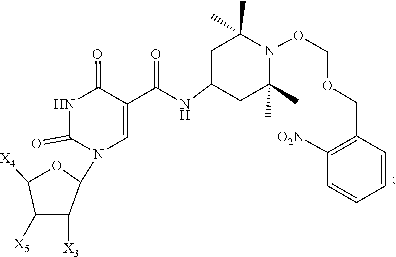
wherein:
X₃ is selected from a methoxy, fluoro, hydrogen, and tert-butyldimethylsilyloxy;
X₄ is selected from OH, —OR′, —SR′, and -Z-P(Z′)(Z″)O—R″, wherein Z, Z′, and Z″ are each independently selected from O and S, R′ is H or a cap, and R″ is H, a cap, or an adjacent nucleotide;
X₅ is selected from —O-ss, —OR′, —SR′, and -Z-P(Z′)(Z″)O—R″, wherein ss is a solid support, Z, Z′, and Z″ are each independently selected from O and S, and R is selected from H and an adjacent nucleotide.

11. An oligonucleotide comprising at least one spin-labeled nucleotide, wherein at least one spin-labeled nucleotide in the oligonucleotide has the structure: 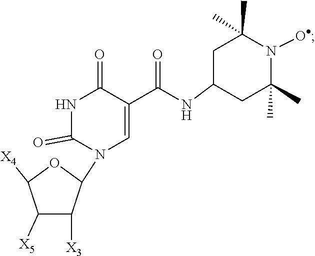
wherein:
X₃ is selected from a methoxy, fluoro, hydrogen, and tert-butyldimethylsilyloxy;
X₄ is selected from OH, —OR′, —SR′, and -Z-P(Z′)(Z″)O—R″, wherein Z, Z′, and Z″ are each independently selected from O and S, R′ is H or a cap, and R″ is H, a cap, or an adjacent nucleotide;
X₅ is selected from —O-ss, —OR′, —SR′, and -Z-P(Z′)(Z″)O—R″, wherein ss is a solid support, Z, Z′, and Z″ are each independently selected from O and S, R′ is H or a cap, and R″ is H, a cap, or an adjacent nucleotide.

12. A method of producing an oligonucleotide of claim 11, comprising synthesizing an oligonucleotide comprising at least one protected spin-labeled nucleotide having the structure: 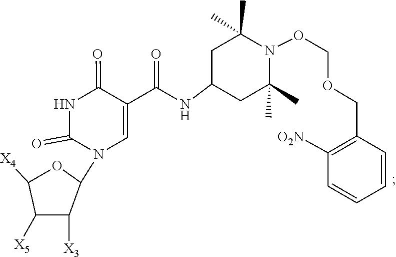
wherein:
X₃ is selected from a methoxy, fluoro, hydrogen, and tert-butyldimethylsilyloxy;
X₄ is selected from OH, —OR′, —SR′, and -Z-P(Z′)(Z″)O—R″, wherein Z, Z′, and Z″ are each independently selected from O and S, R′ is H or a cap, and R″ is H, a cap, or an adjacent nucleotide; and
X₅ is selected from —O-ss, —OR′, —SR′, and -Z-P(Z′)(Z″)O—R″, wherein ss is a solid support, Z, Z′, and Z″ are each independently selected from O and S, R′ is H or a cap, and R″ is H, a cap, or an adjacent nucleotide;
on a solid support; and removing the 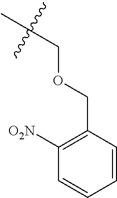 [SMILES: *COCc1ccccc1[N+](=O)[O-]]
protecting group.

13. The method of claim 12, wherein the protecting group is removed by exposing the oligonucleotide to UV light.

14. The method of claim 3, wherein X₁ and X₂ are both methoxy.

15. The method of claim 3, wherein X₃ is hydrogen.
